(19) United States
(12) Patent Application Publication
(10) Pub. No.: US 2018/0140543 A1
(43) Pub. Date: May 24, 2018

(54) PHARMACEUTICAL COMPOSITION

(71) Applicant: National Tsing Hua University, Hsinchu (TW)

(72) Inventors: Hsing-Wen SUNG, Hsinchu (TW); Po-Yen LIN, Hsinchu (TW); Er-Yuan CHUANG, Hsinchu (TW); Ching-Hua CHIANG, Hsinchu (TW)

(21) Appl. No.: 15/797,413

(22) Filed: Oct. 30, 2017

(30) Foreign Application Priority Data
Nov. 18, 2016 (TW) 105137833

(51) Int. Cl. A61K 9/46; A61K 31/12; A61K 31/337; A61K 31/704; A61K 9/00

(52) U.S. Cl. CPC A61K 9/0007; A61K 31/12; A61K 9/0053; A61K 31/704; A61K 31/337

(57) ABSTRACT

The present invention provides a pharmaceutical composition, comprising a hydrophobic active ingredient; a surfactant having hydrophobic end and hydrophilic end; an acidic component; and an effervescent ingredient. The acidic component reacts with the effervescent ingredient in water to generate carbon dioxide. The hydrophobic ends of the surfactant surround carbon dioxide. The hydrophobic active ingredient attaches to the hydrophobic ends of the surfactant.

### BACKGROUND OF THE INVENTION

### 1. Field of the Invention

The present invention relates to a pharmaceutical composition, particularly to a pharmaceutical composition used to transport a hydrophobic active ingredient.

### 2. Description of the Prior Art

Many common hydrophobic drugs, such as curcumin, paclitaxel and doxorubicin, have been proved to have a good therapeutic effect in experiments. However, the hydrophobicity thereof hinders them from mixing homogeneously in fabrication, or makes them hard to disperse while they disintegrate in the digestive organs, or causes them to deposit. Thus, the hydrophobic drugs are hard to be absorbed by living bodies and suffer low bioavailability. The abovementioned problems may affect the therapeutic effect, generate some side-effects, retard extensive clinic application, and impede further development of the hydrophobic drugs. Therefore, hydrophobic drugs are normally administrated in intravenous infusion. In order to avoid the inconvenience of invasive treatment, the current tendency is to develop appropriate carriers for fabricating oral hydrophobic drugs.

The common carriers for oral drugs include liposomes, nanoparticle carriers made of chitosan and γ-polyglutamic acid (γ-PGA), etc. The chitosan and γ-PGA carrier system is characterized in good gastric acid tolerance and dissolvable in the small intestine to release active ingredients. However, the fabrication process of the drugs using the chitosan and γ-PGA carrier system is very complicated and unfavorable for mass production, wherein the ingredients of the drug are mixed and dried in a special process and then enveloped in gelatin capsules. The dissolution of a capsule in the small intestine is usually incomplete and hard to control, which is likely to degrade the effect of drugs. Therefore, an improved carrier of oral hydrophobic drugs should favor the users thereof.

### SUMMARY OF THE INVENTION

One objective of the present invention is to provide a pharmaceutical composition, which comprises a hydrophobic active ingredient; a surfactant having hydrophobic ends and hydrophilic ends; an acidic component; and an effervescent ingredient, wherein the acidic component reacts with the effervescent ingredient in water to generate carbon dioxide, and wherein the hydrophobic ends of the surfactant surround carbon dioxide, and wherein the hydrophobic active ingredient attaches to the hydrophobic ends of the surfactant.

In one embodiment, the surfactant comprises anionic surfactants, cation surfactants, amphiprotic surfactants, or nonionic surfactants.

In one embodiment, the surfactant comprises sodium lauryl sulfate, polyethylene glycol sorbitan monooleate, or sodium dodecyl benzene sulfonate.

In one embodiment, the effervescent ingredient comprises carbonates or bicarbonates.

In one embodiment, the acidic component comprises diethylenetriaminepentaacetic dianhydride (DTPA anhydride), organic acid anhydride, citric acid, or decanoic acid.

In one embodiment, the hydrophobic active ingredient comprises curcumin, paclitaxel, or doxorubicin.

In one embodiment, the pharmaceutical composition is in form of a tablet or a capsule.

In one embodiment, the pharmaceutical composition further comprises an enteric coating enveloping the tablet or capsule.

In one embodiment, the enteric coating comprises a methacrylic acid copolymer, hypromellose phthalate, hydroxypropyl cellulose acetate, hydroxypropyl cellulose succinate, or carboxy methyl ethyl cellulose.

Below, embodiments are described in detail in cooperation with the attached drawings to make easily understood the objectives, technical contents, characteristics and accomplishments of the present invention.

### BRIEF DESCRIPTION OF THE DRAWINGS

The patent or application file contains at least one drawing executed in color. Copies of this patent application publication with color drawing(s) will be provided by the Office upon request and payment of the necessary fee.

FIG. 1 is an ultrasonic image showing that a pharmaceutical composition of the present invention reacts in water;

FIG. 2A is a fluorescent image captured by a confocal microscope and showing that a pharmaceutical composition of the present invention reacts in water;

FIG. 2B is a diagram schematically showing the structure generated in water by a pharmaceutical composition of the present invention;

FIG. 3A is a fluorescent image captured by a confocal microscope and showing that a pharmaceutical composition of the present invention reacts in an interface of water and air;

FIG. 3B is a diagram schematically showing the structure generated in an interface of water and air by a pharmaceutical composition of the present invention;

FIG. 4 shows the results of the drug release experiments of a hydrophobic ingredient in different dosage forms; and

FIG. 5 shows the distributions of a hydrophobic ingredient in different dosage forms in tissues of living bodies.

### DESCRIPTION OF THE PREFERRED EMBODIMENTS

The present invention will be described in detail with embodiments and attached drawings below. However, these embodiments are only to exemplify the present invention but not to limit the scope of the present invention. In addition to the embodiments described in the specification, the present invention also applies to other embodiments. Further, any modification, variation, or substitution, which can be easily made by the persons skilled in that art according to the embodiment of the present invention, is to be also included within the scope of the present invention, which is based on the claims stated below. Although many special details are provided herein to make the readers more fully understand the present invention, the present invention can still be practiced under a condition that these special details are partially or completely omitted. Besides, the elements or steps, which are well known by the persons skilled in the art, are not described herein lest the present invention be limited unnecessarily. Similar or identical elements are denoted with similar or identical symbols in the drawings. It should be noted: the drawings are only to depict the present invention schematically but not to show the real dimensions or quantities of the present invention. Besides, matterless details are not necessarily depicted in the drawings to achieve conciseness of the drawings.

The present invention proposes a pharmaceutical composition, which comprises a hydrophobic active ingredient; a surfactant having hydrophobic ends and hydrophilic ends; an acidic component; and an effervescent ingredient. The surfactant comprises anionic surfactants, cation surfactants, amphiprotic surfactants, or nonionic surfactants. It is preferred: the surfactant comprises sodium lauryl sulfate, polyethylene glycol sorbitan monooleate, or sodium dodecyl benzene sulfonate. The effervescent ingredient may include carbonates or bicarbonates. The acidic component may include organic acids or inorganic acids. The acidic component may be selected from a group including tartaric acid, malic acid, maleic acid, fumaric acid, succinic acid, lactic acid, ascorbic acid, amino acid, glycolic acid, adipic acid, boric acid, potassium hydrogen tartrate, diethylenetriaminepentaacetic dianhydride (DTPA anhydride), organic acid anhydrides, and combinations thereof. The organic acid may include an organic acid anhydride. The organic acid may include but is not limited to include citric acid anhydride, succinic acid anhydride, or another appropriate organic acid anhydride.

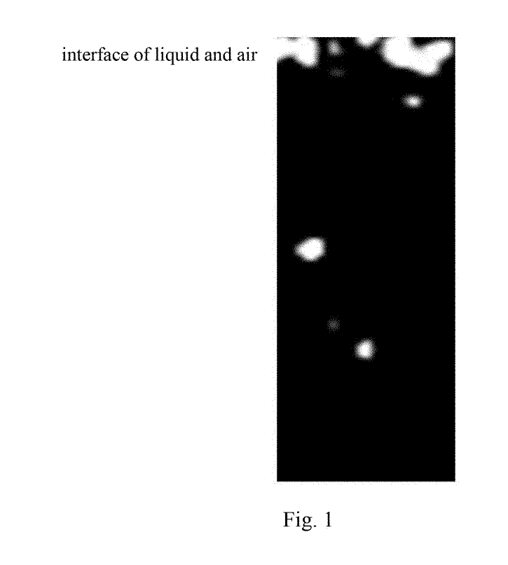

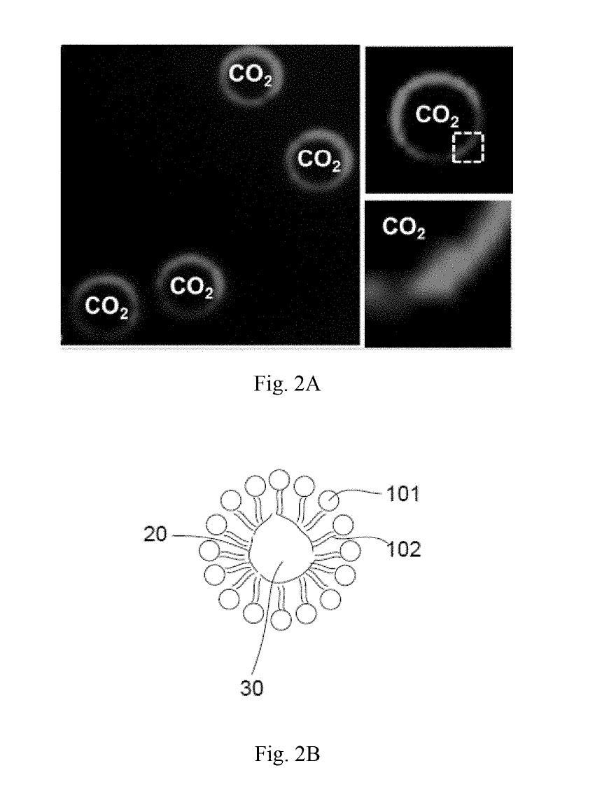

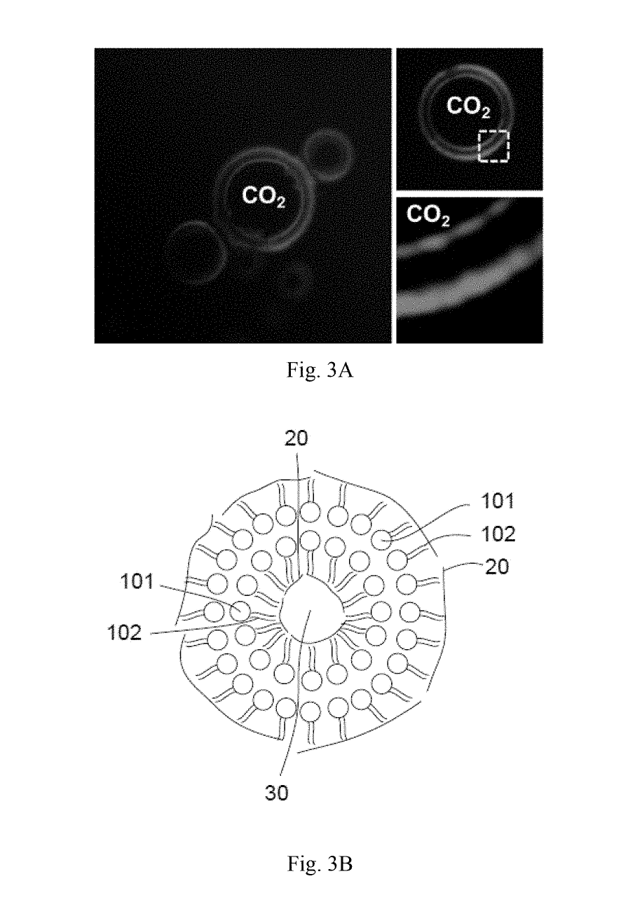

Refer to FIG. 1, FIG. 2A. FIG. 2B, FIG. 3A, and FIG. 3B. While the pharmaceutical composition of the present invention is dissolved in water, the acidic component is dissociated into acid. The acid reacts with the effervescent ingredient to generate carbon dioxide gas. The hydrophobic ends 102 of the surfactant surround the carbon dioxide gas 30 that functions as the gas core. The hydrophobic active ingredient 20 attaches to the hydrophobic ends 102 of the surfactant. Thus are formed monolayer bubble structures, as shown in FIG. 2A and FIG. 2B. While the bubbles shown in FIG. 2A and FIG. 2B approach the surface of water, i.e. the interface of air and water, the hydrophilic ends 101 of the surfactant and the hydrophilic ends 101 of the abovementioned monolayer bubble structures move toward each other and attract mutually. Besides, the hydrophobic active ingredient 20 attaches to the hydrophobic ends 102 of the surfactant. Thus are formed the bilayer bubble structures, as shown in FIG. 3A and FIG. 3B.

The pharmaceutical composition of the present invention may be fabricated into tablets, capsules, or other oral dosage forms. In one embodiment, the pharmaceutical composition of the present invention further comprises an enteric coating, which is sprayed to envelop a tablet, a capsule, or another dosage form. The enteric coating may include a methacrylic acid copolymer, hypromellose phthalate, hydroxypropyl cellulose acetate, hydroxypropyl cellulose succinate, or carboxy methyl ethyl cellulose. While the pharmaceutical composition is swallowed by a living body, the sprayed enteric coating can protect the pharmaceutical composition against the attack of gastric acid in the stomach. After entering the small intestine, the enteric coating is dissolved. Then, the acidic component reacts with water to generate an acidic environment. In detail, the acidic component can locally generate an acidic environment in the neutral environment of the small intestine to make the effervescent ingredient generate carbon dioxide gas, whereby the structures shown in from FIG. 1 to FIG. 3B are created.

It should be noted: according to requirement, the pharmaceutical composition of the present invention may also comprise excipients, carriers, diluents, flavors, sweeteners, preservatives, antioxidants, humectants, buffer agents, release-control components, dyes, adhesives, suspending agents, dispersants, coloring agents, disintegrating agents, film forming agents, lubricants, plasticizers, edible oils, or combinations thereof.

The pharmaceutical composition of the present invention is applied to transport a hydrophobic active ingredient inside a living body. The hydrophobicity makes the hydrophobic active ingredient hard to be dispersed uniformly inside a living body and thus hard to be absorbed by the living body, causing a problem of low bioavailability. In one embodiment, the hydrophobic active ingredient includes curcumin, paclitaxel, doxorubicin, or another active ingredient hard to dissolve in water.

These are always the focuses of medicine research: improving low solubility, transporting instable or high-toxicity medicine, increasing the amount of the medicine transported to the target tissue, and improving the efficiency of transporting macromolecule medicine into cells. Many of anticancer drugs, anti-AIDS drugs, and immunotherapy drugs are bulky polycyclic compounds of low aqueous solubility and feature hydrophobicity. The hydrophobicity assists these drugs to pass through the lipid bilayer membrane and enter into the cells in some extent and increases the specificity of the drugs to special cell receptors. However, the application thereof usually encounters many difficulties. In oral administration, hydrophobic drugs normally have low absorptivity and poor bioavailability. In intravenous administration, hydrophobic drugs are hard to disperse and likely to block blood vessels and respiratory tracts. Besides, low dispersity also causes the drugs to condense in high concentration, which is likely to induce local toxicity in the body and hinder the drugs from entering blood circulation. Thus, the drugs are hard to absorb and low in bioavailability.

The objective of the present invention is to provide a pharmaceutical composition able to effectively transport hydrophobic active ingredients, whereby to overcome the problems encountered in developing hydrophobic drugs. Below, drug-release experiments and animal experiments are used to demonstrate the present invention. In following embodiments, curcumin is used to exemplify the hydrophobic active ingredient and verify the bioavailability of the pharmaceutical composition.

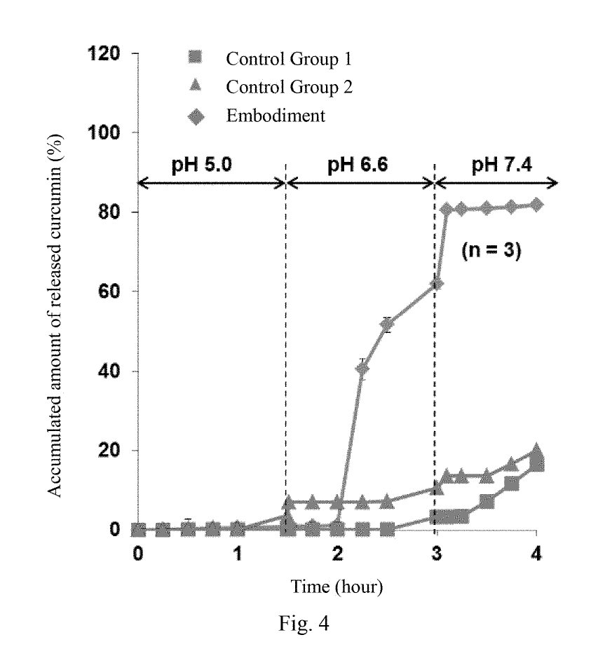

Refer to FIG. 4 for the results of in vitro drug-release experiments for different dosage forms. The embodiment group used in the experiments adopts the pharmaceutical composition containing curcumin as claimed as the present invention. Control Group 1 uses free-form curcumin without any additive. Control Group 2 uses free-form curcumin with sodium hydrogen carbonate (SBC) added. The compositions of the embodiment group and the control groups are all fabricated into capsules. The capsules of each group is placed in a dialysis bag (MWCO 100 kDa), and the pH buffer, which simulates the physiological environment, is used as the dialysis solution. The dialysis bag is placed and persistently oscillated in an oscillation water bath at a constant temperature of 37° C. The dialysis solution is sampled at specified time points. High-performance liquid chromatography (HPLC) is used to detect the drug released by the bubble carriers in different pH environments. It is observed in FIG. 4: after the experiments have been undertaken for 2 hours, the drug release ratio of the pharmaceutical composition of the present invention is significantly higher than that of the compositions of the control groups. Therefore, the pharmaceutical composition of the present invention is proved to have a very high drug release efficiency.

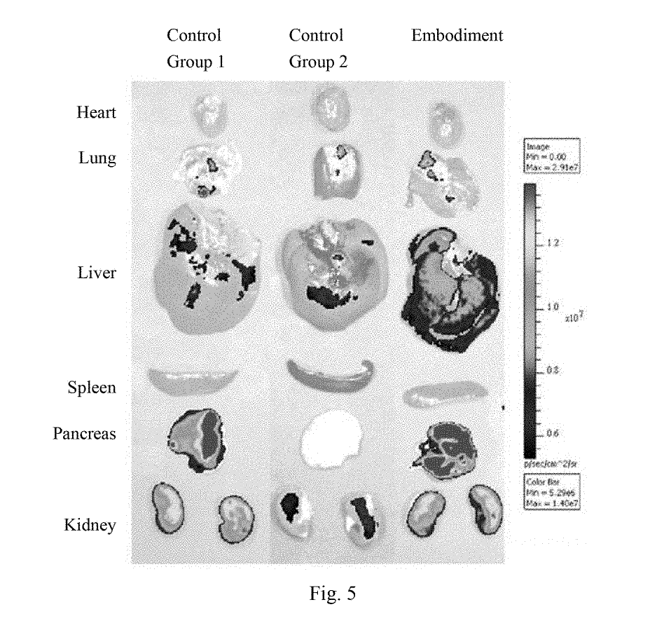

Refer to FIG. 5 showing the distribution of the hydrophobic ingredient of different dosage forms in the tissue of living bodies. Wistar mice (each weighing 300-500 g) are used in the experiments using the in-vivo imaging system (IVIS). In the embodiment of the present invention for the experiments, the curcumin-containing pharmaceutical composition of the present invention is orally delivered with feeding needles to the stomachs of the rat. In Control Group 1, the free-form curcumin is injected hypodermically into the bodies of the rat. In Control Group 2, the free-form curcumin is orally delivered with feeding needles to the stomachs of the rat. After having taken the drugs for 2 hours, the mice are sacrificed with carbon dioxide. The fresh soft tissues of the rat, such as hearts, lungs, livers, spleen, pancreases, and kidneys, are taken out, washed, and placed on the imaging bed. Then, the soft tissues are imaged instantly with IVIS. The tissues and bodies of the rat are handled according to the regulations for experimental animals. The primitive data acquired with IVIS is reconstructed and analyzed with the image reconstruction and analysis software to learn the in-vivo distribution of the multifunctional oral bubble carrier. In the experiments, the molecular imaging system of IVIS is used to assist in positioning the tissues, and the regions of interest (ROI) of the organs/tissues absorbing drugs are manually selected for quantitative analysis. Thus is acquired the absorptivity of each organ/tissue and the pharmacokinetic distribution of the curcumin-containing compositions.

It is found in FIG. 5: in comparison with Control Group 1 (injecting free-form curcumin subcutaneously) and Control Group 2 (delivering free-form curcumin orally), the embodiment orally delivering the curcumin-containing pharmaceutical composition of the present invention has higher absorptivity in livers, pancreases, and kidneys of the rat. Thus is proved that the pharmaceutical composition of the present invention has good bioavailability.

In conclusion, while dissolved in water, the pharmaceutical composition of the present invention generates monolayer bubble structures containing hydrophobic active ingredient and then generates bilayer bubble structures containing hydrophobic active ingredient near the interface of water and air. The abovementioned bubble structures can effectively transport the hydrophobic ingredient to the recipient organs or tissues of living bodies. Further, the release efficiency of the hydrophobic active ingredient of the present invention is higher than that of the conventional dosage form. Therefore, the present invention is highly bioavailable, able to break through the limitation of traditional hydrophobic drugs and provide different directions of drug development.

What is claimed is:

1. A pharmaceutical composition comprising
a hydrophobic active ingredient;
a surfactant having hydrophobic ends and hydrophilic ends;
an acidic component; and
an effervescent ingredient,
wherein the acidic component reacts with the effervescent ingredient in water to generate carbon dioxide; the hydrophobic ends of the surfactant surround carbon dioxide; the hydrophobic active ingredient attaches to the hydrophobic ends of the surfactant.

2. The pharmaceutical composition according to claim 1, wherein the surfactant comprises anionic surfactants, cation surfactants, amphiprotic surfactants, or nonionic surfactants.

3. The pharmaceutical composition according to claim 1, wherein the surfactant comprises sodium lauryl sulfate, polyethylene glycol sorbitan monooleate, or sodium dodecyl benzene sulfonate.

4. The pharmaceutical composition according to claim 1, wherein the effervescent ingredient comprises carbonates or bicarbonates.

5. The pharmaceutical composition according to claim 1, wherein the acidic component comprises diethylenetriaminepentaacetic dianhydride (DTPA anhydride), organic acid anhydride, citric acid, or decanoic acid.

6. The pharmaceutical composition according to claim 1, wherein the hydrophobic active ingredient comprises curcumin, paclitaxel, or doxorubicin.

7. The pharmaceutical composition according to claim 1, wherein the pharmaceutical composition is in form of a tablet or a capsule.

8. The pharmaceutical composition according to claim 7 further comprising an enteric coating enveloping the tablet or capsule.

9. The pharmaceutical composition according to claim 8, wherein the enteric coating comprises a methacrylic acid copolymer, hypromellose phthalate, hydroxypropyl cellulose acetate, hydroxypropyl cellulose succinate, or carboxy methyl ethyl cellulose.
